(19) United States
(12) Patent Application Publication
(10) Pub. No.: US 2007/0231391 A1
(43) Pub. Date: Oct. 4, 2007

(54) Anti-microbial and hydrophilic article and methods for manufacturing the same

(75) Inventors: Ronald L. Bracken, Conyers, GA (US); James M. Lambert, Staunton, VA (US); Gregory J. Mann, Conyers, GA (US)

Correspondence Address: BRIAN M. BURN, ESQ.;C.R. BARD MEDICAL DIVISION, P.O. BOX 52050, c/o PORTFOLIOIP, MINNEAPOLIS, MN, 55402, US

(73) Assignee: C.R. Bard, Inc., Minneapolis, MN (US)

(21) Appl. No.: 11/542,452

(22) Filed: Oct. 3, 2006

Related U.S. Application Data
(60) Provisional application No. 60/723,826, filed on Oct. 5, 2005.

(51) Int. Cl. A61K 9/14

(52) U.S. Cl. 424/486

(57) ABSTRACT

An antimicrobial and hydrophilic article that is microbial resistant comprises an antimicrobial agent and a hydrophilic agent combined with a base material. The combination can be extruded, calandarized, or molded to form the medical article, ensuring that the anti-microbial properties are not localized at the surface. The entire bulk of the article, including the surface thereof, is capable of having anti-microbial characteristics, allowing refreshening of the surface with time.

### CROSS REFERENCE TO RELATED APPLICATION

This application claims priority to U.S. Provisional Application No. 60/723,826 filed Oct. 5, 2005.

### BACKGROUND

The disclosure relates generally to medical articles, for example articles with anti-microbial properties and methods of making the same. The disclosure provides such articles with enhanced antimicrobial properties. Such medical articles, for example, urinary collection containers, catheters and tubing attached thereto, and endotracheal tubes are commonly made of polymer materials such as PVC films or tubing.

Patients in a hospital are generally catheterized either internally or externally, which often results in the risk of urinary tract infections created from microorganism growth within the collection bag and its associated tubing. Recent studies have shown that hospital acquired or “nosocomial” urinary tract infections (UTIs) affect about 900,000 Americans annually. J. R. Johnson, P. L. Roberts, R. J. Olsen, K. A. Moyer, and W. E. Stann, “Prevention of Catheter Associated Urinary Tract Infection with a Oxide-Coated Urinary Catheter Clinical and Microbiologic Correlates, 162 Journal of Infectious Diseases,” 1145-1150 (1990). Many of these UTIs are acquired in hospitals with the result that UTIs account for about 40% of all hospital acquired infections. Of the UTIs acquired in hospitals, about 80% are catheter associated. These hospital-related UTIs were found to prolong hospital stays by an average of 2.4 to 4.5 days and to substantially increase the hospital cost per episode. If the occurrence of catheter-associated UTIs could be reduced, the annual savings in hospital cost alone could be substantial.

One source of catheter related UTIs is suspected to be bacteria progressing from the patient's meatus through the peri-urethral space into the bladder. One method for attempting to prevent UTIs relates to coating the urinary catheter with silver oxide to kill bacteria that may find its way down the patient's periurethral space. Descriptions of methods for preventing pathogens from migrating from a urine collection bag up through a catheter and into the urethra may be found in, e.g., U.S. Pat. Nos. 4,529,398 by Wong; 4,661,100 by Rechsteiner; 5,267,989 by Moyet-Ortiz; 4,863,445 by Mayhan; 4,417,892 by Meisch; and 4,372,313 by Villari, the disclosures of which are incorporated herein by reference.

A common approach to helping prevent UTIs described in the urinary catheter art is to include a sterilizing agent in the catheter or in the collection bag so that pathogens cannot migrate up the catheter. For example, a dispensing device having a polymer with a chemoprophylactic agent can be placed within the collection bag. The dispensing device begins sterilizing liquid in the collection bag immediately upon contact, and the device is designed such that the sterilizing properties continue for an extended period of time. However, in dialysis collection bags, it may be desirable that the sterilization of the liquid not be commenced immediately upon contact with the dialysis collection bag. Furthermore, in dialysis collection bags, it may not be necessary for the sterilization to be conducted for an extended period of time because the bag may be filled in a very short period of time rather than over a period of many hours.

Bacterial growth in medical articles can also cause other problems such as respiratory infections and infections in surgical or catheter sites. Microorganism growth in a respiratory device can travel from the device, for example, through the tubing, into the lungs, and can cause infections such as ventilator-associated pneumonia. Surgical sites can also be infected by bacterial growth, for example, in drainage devices, that can travel from the drainage device into the surgical sites.

There are many drugs, compounds, solutions, and/or materials that show antimicrobial properties, some of which are silver salts, penicillin, sulfa drugs, chlorhexidine, and many others known in the art. However, many of the known antimicrobials are undesirably heat sensitive and can degrade at temperatures that are reasonable and customary for injection molding or extrusion of articles, in particular, urine collection bags. Similarly, some known antimicrobials undesirably discolor when exposed to light or heat. A number of antimicrobials are opaque, which would render otherwise transparent articles opaque or hazy. Furthermore, some antimicrobials have odors that are noticeable to users. A number of antimicrobials are water soluble, which in a urine collection device would leach or dissolve into the urine. This could lower the amount available in the compounded article and potentially weaken, cause pinholes in, or otherwise damage the article.

Furthermore, it is common in the art to extrude or mold articles comprised of hydrophilic materials in order to impart lubricity, swelling of dimensions, or simply to maintain a thin layer of water near the surface of the article. Hydrophilicity can be controlled by the chemical composition of the material. Secondary processes such as plasma deposition or solvent coating are typically employed, rather than compounding hydrophilic agents into other material to impart hydrophilicity where none is present. Whereas this use of hydrophilic materials to produce the article is useful, it can be problematic if swelling of the product is not desirable, for example, when the article needs to have precise dimensions.

Although coating medical devices with antimicrobial agents is widely practiced, it has been determined to have potential drawbacks. For example, it often requires the use of solvents and a process to apply the coating. Also, since the antimicrobial agent is only present in the coating, the antimicrobial properties are localized at the surface upon which the coating is applied. Furthermore, the coating can wear off, resulting in the loss of the desired antimicrobial properties of the device and the risk of infection return.

Accordingly, it is desirable to provide an improved antimicrobial article that overcomes at least some of the drawbacks of the prior art. For example, it would be advantageous to provide an antimicrobial article that exhibits antimicrobial characteristics throughout the entire surface of the article, and to provide an antimicrobial article with a desired clarity, strength or lubricity. In addition, it would be advantageous to provide an improved method of making antimicrobial articles.

### SUMMARY OF THE DISCLOSURE

The present disclosure is directed to an antimicrobial and hydrophilic article and methods of making the same. In particular, the present disclosure is directed to an antimicrobial and hydrophilic article exhibiting antimicrobial and hydrophilic characteristics at the surface, suitably refreshening with time. According to various embodiments, the article comprises a base material with an antimicrobial agent and a hydrophilic agent compounded therein, wherein the compounded material can be extruded, calandarized, or molded into the article. The present disclosure is also directed to methods of making such antimicrobial and hydrophilic articles. The present disclosure is also directed to methods of rendering a rubber or plastic article antimicrobial.

The antimicrobial and hydrophilic article in accordance with the present disclosure generally includes a base material, for example a thermoplastic or thermoset material that is not hydrophilic, such as a polyolefin, silicone, natural or synthetic rubber, or polyvinyl chloride (PVC). The antimicrobial and hydrophilic article can further include an antimicrobial agent and a hydrophilic agent compounded into the base material. The antimicrobial agent is suitably compatible with the base material and can include silver glass compounds and organic acid metal salts. According to various embodiments, the hydrophilic agent is at least partially water-soluble, and can be selected from a wide variety of materials such as PVP, PVP vinyl acetate, hydrophilic polyurethanes, polyethylene oxide (PEO), polyethylene glycol (PEG), acrylics, etc.

According to various embodiments, the present disclosure is directed to compounding an antimicrobially-effective amount of an antimicrobial agent and a hydrophilic agent into the base material, for example prior to thermal processing into final form, to create an article displaying a sufficient amount of antimicrobial and hydrophilic characteristics, rather than merely coating the article. This helps ensure the protection is incorporated throughout the material and furthermore is not subject, or at least not substantially subject, to abrasion or sloughing.

According to one aspect of the present disclosure, the base material is PVC, the antimicrobial agent is a phosphoric acid metal salt, and the hydrophilic agent is a polyethylene oxide (PEO). In one aspect of the present disclosure, the phosphoric acid metal salt is ADK Royalguard® BS-340, which is a commercially-available stabilizing agent from Amfine Chemical Corporation of Allendale, N.J. This stabilizing agent has been found to exhibit antimicrobial properties. The antimicrobial agent can be present in the base material in an amount ranging from about 0.07% to 13.3% by percent wt/wt to the base material, for example from 0.33% to 5%, such as about 3%. The hydrophilic agent can be a PEO having the desired characteristics according to the results desired. For example, the hydrophilic agent can be PEO WSR N-750 (“PEO N-750”), with a molecular weight of about 300,000 and a narrow molecular distribution. PEO N-750 can be present in an amount ranging from, by percent wt/wt to the base material, about 0.1% to about 20%, for example about 1% to about 10%, such as from about 2% to about 5% wt/wt to the base material.

According to another aspect of the present disclosure, the article can comprise a hydrophilic agent and a pre-blended material comprising a base material and an antimicrobial agent. For example, the pre-blended material can be Teknor-Apex PVC, a commercially-available, non-arsenic based antimicrobial PVC, available from Teknor-Apex of Rhode Island, which includes an antimicrobial agent compounded into a base material. The hydrophilic agent can be compounded into the Teknor Apex PVC. According to various embodiments, the Teknor Apex PVC can comprise 100% of the PVC.

Other objects and features of the present disclosure will become apparent from the following description.

The disclosure accordingly comprises the features of construction, combinations of elements, arrangements of parts and methods of operation, which will be exemplified in the constructions and methods hereinafter set forth, and the scope of the disclosure will be indicated in the claims.

### BRIEF DESCRIPTION OF THE DRAWINGS

For a fuller understanding of the disclosure, reference is had to the following description, taken in connection with the accompanying drawing, in which:

FIG. 1 is a graph depicting results of a study conducted to test and compare microbial adherence with materials in accordance with the present disclosure.

### DESCRIPTION

The present disclosure relates to an antimicrobial article and methods of making the same. Suitable non-limiting examples of the anti-microbial article include catheter tubes, urine bags, collection devices, endotracheal tubes, and so forth.

An antimicrobial article in accordance with the present disclosure generally comprises a base material, an antimicrobial agent, and a hydrophilic agent. According to various embodiments, the base material is not hydrophilic and comprises a thermoplastic or thermoset material including polyolefins, natural and synthetic rubber, and silicone rubber. According to various embodiments, the base material is polyvinyl chloride (PVC).

The base material can have an antimicrobial agent compounded therein. The antimicrobial agent is suitably one that is thermally stable for the base material being used. Suitable antimicrobial agents can include silver glass compounds and organic acid metal salts.

According to various embodiments, a suitable antimicrobial agent for use in accordance with the present disclosure is organic acid metal salts, such as salts comprising phosphoric acid esters and zinc metal. According to various embodiments, such materials can provide a stabilizing effect to polymers, for example a heat-stabilizing effect to PVC and other polymer materials used in accordance with the present disclosure. According to various embodiments, the antimicrobial material is ADK Royalguard® BS-340, which is a commercially-available organic acid metal salt stabilizer from Amfine Chemical Corporation.

ADK Royalguard® BS-340 comprises a zinc phosphoric acid ester that can provide several benefits over silver-based antimicrobial compounds. This, and compounds like it, are similar to silver compounds in their antimicrobial properties, but may be less likely to discolor under heat and light and provide other benefits. They can also be effective against a wide range of microorganisms.

As can be appreciated, there are a number of known antimicrobial compounds that may not be able to withstand the heat involved in injection molding or extrusion or heat welding. There are also a number of antimicrobial compounds that may adversely affect the desirable properties of the articles such as clarity, strength, bond ability, etc. There are also a number of compounds that may not be effective against a sufficient number of microorganisms. The phosphoric acid metal salts of the present disclosure, such as ADK Royalguard® BS-340, appear to provide a balance of beneficial properties with little or none of the undesirable properties.

It is also anticipated that there are several compounds that are related but not the same chemically as ADK Royalguard® BS-340 and that there are other compounds that are similar or have similar desirable properties and these could be used singly or in conjunction with others in the same or other articles. It is also appreciated that the ADK Royalguard® BS-340 is low in odor and is substantially insoluble in water so that when in contact with a liquid such as, for example, urine, it will not leach out and become unusable or leave voids in the article.

One desirable aspect of ADK Royalguard® BS-340, and other compounds like it, is that they can be compounded in the various materials, e.g., natural or synthetic rubber, silicone, various polymers, such as PVC or vinyl, polyethylene, polypropylene, ethylene vinyl acetate, metallocene catalyzed polyethylene, or blends thereof, etc., before the article is molded or extruded, and the material will not significantly affect the desirable properties of the article, e.g., drainage tubing can still be solvent bonded, the film can still be RF or heat welded, it would not be toxic, significant amounts would not leach out, and the article or product would remain antimicrobial for extended periods of time.

According to various embodiments, the antimicrobial agent is provided in a concentration that will provide a desired antimicrobial effect. For example, suitable concentrations of the antimicrobial agent can range from about 0.07% to 13.3% wt/wt to the base material (e.g. PVC), such as from about 0.33% to 5%, for example about 3% wt/wt to the PVC.

The base material can also have a hydrophilic agent compounded therein, either simultaneously or in a step independent of the addition of the antimicrobial agent. According to various embodiments, the hydrophilic agent is compatible with the base material being used and can be selected from a wide variety of materials including PVP, PVP vinyl acetate, hydrophilic polyurethanes, polyethylene oxide (PEO), polyethylene glycol (PEG), and acrylics. The hydrophilic agent can be selected according to the effects on properties desired, such as clarity, lubricity, strength, etc. In accordance with one aspect of the present disclosure, the hydrophilic agent is a PEO, which provides a desirable visual product, for example a Polyox™ PEO WSR N-750, available from The Dow Chemical Company of Michigan, U.S.A. The hydrophilic agent is present in an amount ranging from about 0.1 to 50% by weight, relative to the weight of the base material; according to another aspect of the present disclosure, the hydrophilic agent is present in an amount ranging from about 5 to 25%, and 1 to 10% if the hydrophilic agent is PEO; from example, about 10 to 15%, and 2 to 5% if the hydrophilic agent is PEO.

It can be desirable to compound both the hydrophilic agent and the antimicrobial agent into the base material because the combination of the two agents substantially reduces the microbial adherence on the article formed. The reduction in microbial adherence is not simply additive. Rather, there is a dramatic decrease when both are present, as evident from the example given below.

It is also appreciated that these types of compounds can be provided as powders that can be easily handled and mixed into resins before forming into useful articles, and that these types of agents have low toxicity to humans and animals such that workers handling the compound will not be harmed and upon disposal of the article the agent will not leach or harm animals and marine life in the environment. It is also appreciated that these agents are heat stable and can endure injection molding and extrusion temperatures in excess of 250-350° F., and even over 500° F.

According to various embodiments, antimicrobials and hydrophilic agents suitable for use in the present disclosure include those that (1) are heat stable and will not substantially degrade, will not discolor or substantially discolor, (2) are capable of at least substantially maintaining the transparency of the article in which they are incorporated, (3) do not have an odor associated with them, and/or (4) are at least substantially water insoluble, thereby providing a suitable antimicrobial effect over time. Suitable antimicrobials have a wide spectrum of activity (against gram positive, gram negative and yeasts), they produce little or no discoloration, when exposed to heat, light, radiation or moisture, and they are inexpensive and easy to manufacture.

The article in accordance with the present disclosure can be any suitable article such as, for example, those used in the medical field. Non-limiting examples of such articles can include catheters (e.g.,, rectal catheters and urinary catheters), drain bags, cannulae, stents, implant devices, peristaltic pump chambers, endotracheal tubes, wound drains, gastroenteric feeding tubes, arteriovenous shunts, oxygenator and kidney membranes, gloves, wound dressings, catheter securement devices.

A method and an article in accordance with the present disclosure were evaluated in studies and the following example is illustrative of the tests that were conducted. The following example is intended to further illustrate the present disclosure, but is not intended to be construed to limit the scope of the disclosure.

### EXAMPLE 1

The following is one method of producing an article that possesses antimicrobial and hydrophilic properties in accordance with an aspect of the disclosure:

A. Compound the antimicrobial agent and hydrophilic agent into the PVC resin:

Royalguard® BS-340 and PEO (N-750) were blended into the PVC resin pellets at a concentration of 3% wt/wt Royalguard® to the PVC and 2.5% wt/wt PEO (N-750) to the PVC. This was done via tumbling the powders with the pellets, coating the outside of the pellets, passing the resin and powders through an extruder and forming new pellets containing Royalguard® and PEO (N-750). As an alternative, it is possible to compound the powders with additives (i.e. colorants, stabilizers, inhibitors). This would allow the elimination of a separate process step since a colorant, stabilizer, or other additive is typically added at this point.

B. Extrude an article out of the PVC resin with Royalguard® and PEO (N-750):

Typical extrusion temperatures (and typical calendaring temps) are 330-345° F., which is at or about the melt temperature of the resin. The Royalguard® and PEO (N-750) containing pellets were placed into an extruder. The pellets were taken from room temperature through a ramp up temperature to 200-250° F. in zone 1 to 250-300° F. in zone 2 to 300-350° F. in zone 3. If there are other zones in the extruder they can be kept at 300-350° F. The extruder was then used to form the article by extruding molten PVC through a crosshead containing a pin and die to form a tube.

### EXAMPLE 2

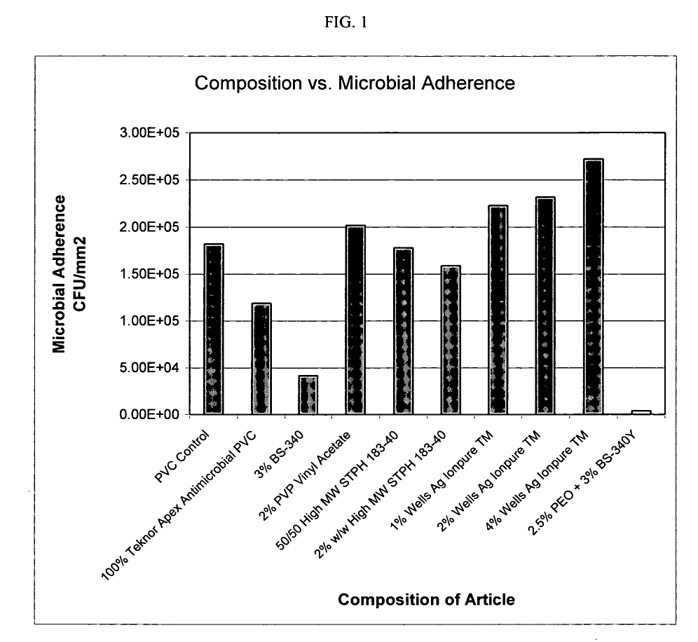

A controlled study on microbial adherence was performed on the product of Example 1 containing both an antimicrobial agent and a hydrophilic agent. The study also involved testing other PVC based articles, namely a PVC control containing no agents, and articles containing either an antimicrobial agent or a hydrophilic agent, singly, in various amounts. In the study, each article was tested for microbial adherence five times, and the average result is presented below in Table 1 and in FIG. 1.

Product manufactured per Example 1 in comparison to other compositions.

TABLE 1

| Composition | Average CFU/mm2 |
|---|---|
| Control without any agents: | |
| PVC Control | 1.82E+05 |
| PVC plus antimicrobial agent: | |
| 100% Teknor Apex Antimicrobial PVC | 1.19E+05 |
| PVC plus 3% BS-340 | 4.16E+04 |
| PVC plus hydrophilic agent: | |
| 2% PVP Vinyl Acetate | 2.02E+05 |
| 50/50 High MW STPH 183-40 | 1.78E+05 |
| 2% w/w High MW STPH 183-40 | 1.59E+05 |
| 1% Wells Ag Ionpure ™ | 2.23E+05 |
| 2% Wells Ag Ionpure ™ | 2.32E+05 |
| 4% Wells Ag Ionpure ™ | 2.72E+05 |
| PVC plus both antimicrobial agent | |
| and hydrophilic agent: | |
| 2.5% PEO + 3% BS-340Y (Example 1) | 3.83E+03 |

The above table indicates that the reduction in microbial activity resulting from the addition of both Royalguard® BS-340 and PEO (N-750) is not merely additive. Instead, there was a 2 log reduction in microbial adherence from the PVC control to Example 1, when an antimicrobial agent and a hydrophilic agent were added to the PVC.

As evident in FIG. 1 and Table 1, the addition of both antimicrobial and hydrophilic agents into the base material resulted in a significant reduction of microbial activity. When an antimicrobial agent was added to the base singly, as shown by 100% Teknor Apex Antimicrobial PVC, a non-arsenic based antimicrobial PVC, as well as by PVC plus BS-340, microbial adherence decreased in comparison to the PVC control. However, when Ionpure™, a commercially-available soluble silver silicate glass from Wells Plastic, was added to PVC, microbial activity did not decrease in comparison to the PVC control. Rather, microbial activity increased compared to the PVC control and continued to increase as more Ionpure was added, as is observable from the chart of FIG. 1.

The addition ofjust a hydrophilic agent, for example, 2% Vinyl Acetate, also did not reduce microbial adherence compared to the PVC control but increased it instead. The microbial adherence of 2% Vinyl Acetate was 2.0E4 greater than the PVC Control. Similarly, the addition of High MW STPH 183-40, a hydrophilic polyurethane urea designed for extrudable coatings, also increased microbial activity when added alone to PVC. Although the presence of certain antimicrobial agents alone reduced microbial adherence in the PVC base article, the hydrophilic agents alone did not. Therefore the collective antimicrobial effect of the two agents added singly to the PVC did not add up to that of the antimicrobial and hydrophilic agents added together. The addition of both an antimicrobial agent and a hydrophilic agent, e.g. represented by 2.5% PEO+3% BS-340Y, forming articles in accordance with the disclosure creates a synergistic reduction in microbial adherence, rather than merely an additive effect of adding each singly.

The present disclosure provides more than the synergistic reduction in microbial adherence. Because the antimicrobial and hydrophilic agents are added prior to thermal processing into final form, the secondary operations and additional solvents or chemicals required in coating the article is eliminated. This secondary operation can be costly and time consuming and leave solvent residues, which must be removed. The present disclosure disposes of the need for the secondary operation and thereby reduces cost and time. Additionally, since the antimicrobial and hydrophilic agents are added prior to processing into final form, the present disclosure also provides protection throughout the material and is not subject to abrasion or sloughing issues. Thus, while there have been shown and described and pointed out fundamental novel features of the disclosure as applied to preferred embodiments thereof, it will be understood that various omissions and substitutions and changes in the form and details of the disclosed disclosure may be made by those skilled in the art without departing from the spirit of the disclosure. It is the intention, therefore, to be limited only as indicated by the scope of the claims appended hereto.

What is claimed is:

1. A medical article that is resistant to microbial adherence, formed by combining materials comprising a base material, an antimicrobially effective amount of an antimicrobial agent, and a hydrophilically effective amount of a hydrophilic agent.

2. The article of claim 1, wherein said antimicrobial agent is compounded into said base material before the material is formed into the article.

3. The article of claim 1, wherein said hydrophilic agent is compounded into said base material before the material is formed into the article.

4. The article of claim 1, wherein said base material comprises a polymer material.

5. The article according to claim 1, wherein said base material comprises a thermoplastic material.

6. The article according to claim 1, wherein said base material comprises a thermoset material.

7. The article according to claim 1, wherein said base material comprises a polyolefin.

8. The article according to claim 1, wherein said base material comprises a natural rubber, a synthetic rubber, or a combination thereof.

9. The article according to claim 1, wherein said base material comprises a silicone rubber.

10. The article according to claim 1, wherein said base material comprises polyvinyl chloride.

11. The article according to claim 1, wherein said antimicrobial agent comprises silver glass compounds.

12. The article according to claim 1, wherein said antimicrobial agent comprises a phosphoric acid metal salt component.

13. The article according to claim 12, wherein said phosphoric acid metal salt component comprises a zinc phosphoric acid ester.

14. The article according to claim 2, wherein said hydrophilic agent is compounded into said base material before the material is formed into the article.

15. The article according to claim 1, wherein said hydrophilic agent comprises a polyvinylpyrrolidone.

16. The article according to claim 1, wherein said hydrophilic agent comprises a polyvinylpyrrolidone vinyl acetate.

17. The article according to claim 1, wherein said hydrophilic agent comprises a polyurethane.

18. The article according to claim 1, wherein said hydrophilic agent comprises a polyethylene oxide.

19. The article according to claim 1, wherein said hydrophilic agent comprises a polyethylene glycol.

20. The article according to claim 1, wherein said hydrophilic agent comprises an acrylic.
